(19) United States
(12) Patent Application Publication
(10) Pub. No.: US 2005/0002971 A1
(43) Pub. Date: Jan. 6, 2005

(54) Cosmetic composition comprising a fatty acid material and preparation method therefor

(76) Inventors: Maria Teresa Belmar, Bedford (GB); Alan Patrick, Bedford (GB); Shiping Zhu, Bedford (GB)

Correspondence Address: UNILEVER;PATENT DEPARTMENT, 45 RIVER ROAD, EDGEWATER, NJ, 07020, US

(21) Appl. No.: 10/496,863

(22) PCT Filed: Nov. 6, 2002
(86) PCT No.: PCT/EP02/12363

(30) Foreign Application Priority Data
Nov. 30, 2001 (EP) 01310051.6

(51) Int. Cl. A61K 7/00

(52) U.S. Cl. 424/401

(57) ABSTRACT

A method of providing a topical base composition for use in the preparation of a topical skin care composition, comprising: (a) providing a cosmetic container; (b) providing in the container an effective amount of a fatty acid material having a melting point in the range of 40° C. to 80° C.; and other components necessary to form a fatty acid soap material which is substantially anhydrous (c) providing sufficient heated water to the container such that substantially all of the fatty acid material is solubilised to provide a fatty acid soap material; and(d) agitating the contents of the container The container comprising an anhydrous composition according to (b), the additional component being a base or a soap, is also claimed.

This invention relates to cosmetic compositions, and to preparation methods therefor. In particular, it relates to base compositions which may be used in cosmetic compositions such as skin and vanishing creams which may be rapidly prepared, for example, directly by a user.

Cosmetic compositions such as skin creams or lotions, and vanishing creams are well known. Such compositions are typically prepared on an industrial scale in a centralised manner, using large-scale equipment. In such a typical production process, for the production of products which are typically emulsions, the highest melting point ingredients are typically heated in a large container until they are molten (typically at around 80° C.); stirred, and further ingredients are added to the composition as it is continually stirred and cooled. The last of these ingredients is often perfume, which may be added when the composition is typically at around 40° C. Thereafter, the composition is cooled and dosed into retail containers. The time and speed of shearing used in the process typically effects the droplet size of the resulting emulsion.

WO-A-91/07943 discloses a typical industrial process wherein all ingredients are mixed with water such that they form a stable oil in water emulsion.

A number of problems may be associated with such large scale methods of manufacture. Foremost of these is the difficulty of individualisation or customisation of products, with the result that typically only relatively few products may be provided to the consumer. An example of this is the time and cost associated with changing products on a production line; because of the need to thoroughly clean the production apparatus between production runs of different products, especially with large scale manufacturing equipment, this contributes significantly to the difficulties of producing many different products on such a line within a reasonable time scale.

As a consequence of this, where a product is a customised or individualised product, and the batch of that particular product is relatively small, the cost of the product is relatively high.

In addition, a typical skin cream composition may easily comprise 60-90% water. As such, water makes up a large proportion of the weight of a topical product. It therefore represents a large proportion of the cost of transporting such a commercial product between manufacture and retailing.

GB-A-633065 discloses a process wherein saponifiable fat or fatty acid is mixed with an alkaline solution, heated and agitated to approximately 90° F. and then further heated to 300-350° F. and maintained at this temperature until a dry powder like substance is formed. This process is energy and time consuming and requires a reaction to take place between fatty acid and base before a base composition is formed. The inefficiency of the process makes it unsuitable for use nowadays.

U.S. Pat. No. 1,500,276 discloses a base in the form of an unctuous mass is prepared containing a neutral soap, water and free fatty acid finely and uniformely dispersed therethrough. The mass comprises water and is of a jelly like consistency. The initial base mixture is dependent on the presence of water and the resulting jelly is unlikely to be of the desired consistency to use for preparing a cosmetic composition in an efficient, instant manner.

Also, in many countries where incomes are relatively low, a consumer often cannot afford to purchase a large container of a cosmetic product. As such, there is high demand for cosmetic compositions which are provided in relatively small dosages, provided these can be made available at relatively low cost.

Yet a further problem associated with topical products is the instability of some of the more desirable ingredients of such products. An example of such an ingredient is retinol. Because retinol is relatively unstable, this leads to the situation where products containing retinol may have a relatively short shelf life, or alternatively a reduction in the functionality of retinol is experienced and accepted during the lifetime of the product. Where topical compositions do contain unstable components, it would be an advantage to be able to prepare them freshly.

We have found a method of manufacturing care compositions for topical application to the skin (such as, for example, skin creams, lotions, vanishing creams, sun tan lotions, moisturisers, and so on) which particularly lends itself to production of relatively small amounts of such compositions in a short time (eg. a few minutes).

In its broadest aspect, it facilitates the manufacture of a base composition, especially in small amounts, which can be readily used to provide all manner of cosmetic compositions, especially topical skin care compositions. Such a base composition can readily be customised by the addition of suitable active and emotive ingredients, to provide topical skin care compositions as required by the consumer. Such “small scale” production can encompass small industrial scale manufacture by a producer, or reconstitution by a consumer of a small amount of topical product from a concentrate.

Although the use of the base composition on small scale is highly preferred it may also readily be used in an efficient larger scale process such as at industrial scale.

Thus, according to a first aspect, there is provided a method of providing a base composition for use in the preparation of a cosmetic composition, especially a topical skin care composition, comprising:
(a) providing a cosmetic container;
(b) providing in the container an effective amount of fatty acid material having a melting point in the range 40-80° C. and any other components necessary to form a fatty acid soap material which is substantially anhydrous;
(c) providing sufficient heated water to the container such that substantially all of the fatty acid material is solubilised to provide a fatty acid soap material; and
(d) agitating the contents of the container;
   whereby a cream or lotion base is formed.

The provision of an effective amount of fatty acid soap material in the container can take a number of different forms.

In one embodiment, the fatty acid soap material can be provided in the container by simply pre-dosing the container with a mixture of soap and fatty acid material. The fatty acid soap material is formed on the addition of hot water. In a further preferred embodiment, the fatty acid soap material may be provided from an unreacted mixture of fatty acid material and an effective amount of a suitable organic or inorganic base which is pre-dosed into the container. On the addition of hot water to this mixture, a fatty acid soap material is formed in situ by partial saponification of the fatty acid.

By “solubilised” in this context we mean a possible combination of dissolving and/or melting of the fatty acid.

The provision of “heated water” in the container may also take several forms. In its simplest aspect, the heated water may be provided by simple dosing into the container of hot (eg. boiled) water. However other methods are envisaged; specifically envisaged is the dosing into the container of cold water, which is subsequently heated either by the application of microwave energy (from eg. a domestic microwave oven), or the application of ultrasound as a heating and mixing source.

It is an advantage of the claimed method that water is only added after a base comprising fatty acid soap material has been formed. Hence in step (b) the composition in the container is essentially anhydrous enabling low cost transport and long keepability.

Conveniently, the essentially anhydrous composition in the container is a simple mechanical mixture, and may conveniently be in the form of a tablet, powder or amorphous mass. Hence in another form of the invention, there is provided an anhydrous composition in the form of a tablet, powder or amorphous mass for dosing into a container, for use according to the invention. Such an anhydrous composition may be provided separately to the container in which preparation of the composition occurs. Each anhydrous composition may be individually wrapped.

Where the container is a cosmetic container and the preparation is being carried out for the preparation of an individual portion of the skin care composition by a consumer, it will be necessary to seal the cosmetic container prior to agitation. Such cosmetic containers may preferably have a volume of 20-250 ml, more preferably 25-100 ml, even more preferably 25-50 ml.

Even then, it is preferred that in such containers the volume of contents (eg. water, base materials, and other components necessary to make the topical composition) do not make up more than 50% of the volume of the container, so as to allow sufficient room for thorough mixing of the contents during agitation.

The optimum size and shape for the container can easily be determined by routine simple experimentation. However, the optimum container will provide for both rapid and thorough solubilisation by the heated water of the fatty acid material, but also rapid heat loss. This second feature is significant, since ideally agitation of the container is continued until the contents of the container are “set”, ie. they do not readily splash around the container during shaking.

The dimensions of the “container” are significant in ensuring that thorough solubilising of the fatty acid material occurs. When the embodiment of the invention lies in the provision of a container to the consumer which contains the anhydrous base ingredients and other components necessary for a topical skin composition, this will have consequences for the dimensions and size of the container. However, given that the invention is also suitable for (small scale) industrial production, the “container” and its dimensions may relate to the appropriate mixing chamber in a batchwise or continuous mixing apparatus. In particular, as well as the making up of individual compositions according to the invention by the user at home, the invention also lends itself to the (small scale) manufacture of compositions for example at the point of sale in shops and beauty parlours, using pilot plant machinery, and in mobile vending facilities which are common in various parts of the world. According to a further aspect of the invention, there is provided a re-sealable cosmetic container containing an anhydrous composition comprising a fatty acid material having a melting point in the range 40° C. to 80° C., and an organic or inorganic base.

According to yet a further aspect, there is provided a re-sealable cosmetic container containing an anhydrous composition comprising a mixture of soap and a fatty acid material, the fatty acid material having a melting point in the range 40° C. to 80° C.

By “anhydrous” in this context is meant containing less than 5% by weight of water, preferably less than 2% water, even more preferably less than 1% water, and ideally less than 0.5% water.

The method and packaged composition of the invention provide a surprisingly effective method of manufacturing a skin composition base for a topical application, which base material lends itself both to rapid small scale production, and also customisation with such further ingredients or additives as may be necessary to provide the desired effective topical composition. Such extra ingredients necessary for the formulation of the final topical composition may conveniently (but not essentially) be pre-dosed into the container along with the fatty acid material and other fatty acid soap ingredients.

It is preferred that the base composition resulting in step (b) is taken to a remote location before the addition of water to form the lotion or cream base. In case the composition of step (b) is applied in a (small scale) industrial process the composition may be stored as such and used in a further process and transported to a separate vessel for continuation with step (c).

Although not wishing to be bound by theory, the essence of the invention is thought to lie in the provision of heated hot water to a mixture of ingredients including a fatty acid material to provide a fatty acid soap material, which fatty acid soap material combined with the water provides a base for a cream or lotion which has desirable physical and rheological properties. In practising the method of the invention, the amount and temperature of the heated water added to the container should be sufficient to cause substantially all of the fatty acid material in the container to melt and/or dissolve. This facilitates the formation of the appropriate crystal structure, to provide the base with the desirable Theological properties.

The small scale nature of the invention is important and therefore preferred, since it is especially by the provision of a relatively small container that substantially all of the fatty acid material can be readily melted and/or dissolved using only heated water, and secondly that the ingredients of the topical composition can be sheared by a regime (e.g. shaking) which does not require specialist shearing equipment. Thirdly, the cooling speed of the product is fast without using complicated equipment.

That said, in certain embodiments it may be possible and indeed desirable to use shearing equipment.

A further important aspect of the invention lies in that the relative amounts of ingredients of the fatty acid soap material and water are determined and used to ensure the formation of the so-called “fatty acid soap material”. The fatty acid soap material has a different structure from either fatty acid material or soap. “Pure” fatty acid material is thought to have a closely packed bilayer structure such that water cannot readily penetrate the bilayers. This contributes to the relative insolubility in water of the fatty acid material. Fatty acid soap material also has a bilayer structure but with the bilayers relatively widely spaced, which allows water molecules more readily between the bilayers and increases the solubility.

The resulting partially hydrated fatty acid soap material provides desirable rheological properties for the resulting base product, which properties can be manipulated inter alia by varying the relative amounts of the starting ingredients, and hence the relative amounts of fatty acid material and soap in the fatty acid soap material.

There are other methods of manipulating the final properties of the compositions according to the present invention. For example, in an envisaged aspect, if the container is subjected to ultrasound as part of the agitation step, it has been found that the resulting cosmetic product is slightly harder than otherwise.

To ensure formation of the appropriate crystal structure and sufficient melting/dissolving of the fatty acid soap material ingredients, and also avoid premature reaction, it is preferable that the ingredients of the fatty acid soap material be substantially anhydrous prior to the provision of heated water. Once the heated water has been provided to the container, agitation of the water and the fatty acid soap material ingredients is required for homogeneity. Where the container is a re-sealable container in which a consumer is making an individual portion of skin composition, that agitation is conveniently provided by shaking the sealed container, which should provide sufficient agitation.

Conveniently, the container is agitated for a period of at least 10 seconds, more preferably at least 20 seconds, even more preferably at least 30 seconds. Conveniently agitation may last up to 5 minutes. Conveniently, agitation is continued until at least the time that the contents no longer splash around the container during agitation.

It is also important that the temperature and amount of the heated water used be sufficient to melt and/or dissolve substantially all of the fatty acid material in the container. The minimum temperature of the hot water required will depend, amongst other things, on the amount of water used, but the heated water will typically have a temperature of at least 80° C., preferably at least 85° C., more preferably at least 90° C., even more preferably at least 95° C. and may even be boiling.

In the embodiments in which the soap component of the fatty acid soap material is formed in situ, especially when the ingredients are fatty acid material and base, by controlling the ratio of the fatty acid material to base, and also the amount of water added, it is possible to control the degree of saponification of the fatty acid material. Depending on the ratio of the unneutralised fatty acid material to soap, and also the amount of water present, it is possible to manipulate the crystal phase structure of the resulting mixture, which mixture has been found to be a particularly suitable base material for topical skin compositions.

An essential element of the invention is the fatty acid material. “Fatty acid material” may include blends of fatty acids, typically containing fatty acid moieties with chain lengths of from C₈ to C₂₂. The fatty acid material may also contain relatively pure amounts of one chain length fatty acid moiety. Suitable fatty acids from which fatty acid soaps may be derived include pelargonic, lauric, myristic, palmitic, stearic, isostearic, oleic, linoleic, ricinoleic, arachidic, behenic and erucic acids. Although normally saturated, suitable fatty acid materials may contain unsaturated fatty acid moieties, and may contain fatty acid moieties having a degree of substitution, such as e.g. hydroxy fatty acids. Melting points of the individual fatty acids are also relatively unimportant; what is most important is the overall melting point of the fatty acid material, which should be in the range of 40° C. to 80° C.

In certain preferred embodiments, the fatty acid material will contain relatively high amounts (e.g. at least 50%, preferably at least 75%) of stearic and/or palmitic acid moieties. The chain length of the fatty acid material used according to the invention will influence the minimum temperature of the heated water needed to be used in the invention, since an important aspect of the invention is the fairly thorough melting of the fatty acid material on the provision of the heated water. However, the chain length of the ingredients of the fatty acid material also determines the Theological properties of the resultant skin composition base. A fatty acid material mix containing relatively high proportions of stearic and palmitic acid moieties has been found to be particularly suitable for use for manufacturing skin creams and lotions which may be used in temperate to hot climates. However, fatty acid soap material mixtures containing relatively high amounts of lower chain length fatty acid moieties (e.g. more than 50% of the fatty acid moiety having a chain length of C₈-C₁₄) may also be suitable for the preparation of skin compositions for use in relatively cold climates.

Topical cosmetic compositions according to the invention typically have a pH in the region 6 to 9, preferably 6.5 to 8.

In certain embodiments, a further important element of the invention is the base material. A suitable base may be either organic or inorganic. Preferred bases include inorganic bases such as hydroxide materials, such as sodium or potassium hydroxide, as well as organic bases such as triethanolamine. It has been found that variation of the base used can influence the sensory properties of the resulting cream or lotion.

The amount of base added to the composition will be such as to provide a satisfactory cream or lotion base composition on the provision of heated water. The amount of base added is so as to typically provide partial saponification of the fatty acid material thereby generating fatty acid soap material, with it being possible to adjust the Theological properties of the resultant base composition depending on the amount of base added.

Base compositions formed according to preferred embodiments of the invention contain in the fatty acid soap material both unneutralized fatty acid material and soap, the ratio of the two being carefully controlled to optimise physical and sensory properties. A cosmetic base comprising fatty acid soap material which contains too much fatty acid material, or too little soap will typically be too hard to spread on the skin, not be in the form of a viscous but flowable cream, and will probably have a grainy feel. However, too much soap or too little fatty acid material in the base composition could produce a composition which feels too slimey.

Typically, the ingredients of the fatty acid soap material are adjusted so as to provide an acid soap composition comprising a weight ratio of soap to fatty acid material after heated water has been provided of 1:1 to 1:20, more preferably 1:2 to 1:10, even more preferably 1:5 to 1:10. In certain embodiments, particularly suitable skin care composition bases have been achieved when the ratio of soap to fatty acid material is around 1:7.

In other embodiments, a preferred starting ingredient of the fatty acid soap material will be a soap itself By “soap” is meant an alkali metal, ammonium salt (usually a sodium or potassium salt) of a long chain (typically C₈ to C₂₂) fatty acid. Preferred soaps have monovalent cations.

In a preferred embodiment, the invention may comprise a re-sealable cosmetic container containing a “concentrate”, which may be dry, and may conveniently be in the form of a tablet, dried powder or amorphous mass, which concentrate is for the provision of a skin care composition after reconstitution with hot water. The concentrate may contain the essential base materials, i.e. a fatty acid material and an organic or inorganic base, or soap, and any other additives which are necessary or desirable to form the eventual topical skin care cream or lotion.

Compositions according to the invention are particularly suited as base materials to provide skin care creams or lotions. In some circumstances, the resultant fatty acid soap material formed after reconstitution could provide a suitable topical skin care composition on its own.

However, it is normally desirable to add further ingredients (also referred to as additives) to the composition to provide the final skin care composition.

Preferred cosmetic products are skin care compositions and vanishing creams. The amount of gas in these products is preferably below 50% of their total volume, more preferred below 25%, most preferred below 5%.

Examples of additives suitable for cosmetic compositions, especially skin case compositions are given below.

Besides water, relatively volatile solvents such as e.g. C₁₋₃ monohydric alcohols may be part of the cosmetic vehicle of composition, though they are usually present only at relatively low levels since they tend to evaporate when hot water is added to the base concentrate.

Resulting topical compositions according to the invention may typically contain 50% to 90% water, more preferably 70% to 85% water, and sufficient fatty acid, base and/or soap so as to provide a suitable level of fatty acid soap material so as to structure the product. Suitable levels of fatty acid may be in the range 5 to 25%, more preferably 10% to 20%, base in the range 0.1 to 5% (if present) and soap 0.1 to 10%, more preferably 1% to 4% (if present).

Topical cosmetic compositions according to the invention typically comprise 60-99% of aqueous cosmetic base (eg. water, fatty acid and fatty acid base), the balance comprising other components necessary to provide the desired form of topical skin care composition.

Emollient materials may also serve as cosmetically acceptable additives. These may be in the form of silicone oils and synthetic esters. Amounts of the emollients may range anywhere from 0.1 to 25%, preferably between 1 and 20% by weight of the final topical skin care composition.

The emollient material may be a silicone oil, an ester or a mixture of these. Silicone oils may be divided into the volatile and non-volatile variety. The term “volatile” as used herein refers to those materials which have a measurable vapour pressure at ambient temperature. Volatile silicone oils are preferably chosen from cyclic or linear polydimethyl siloxanes containing from 3 to 9, preferably from 4 to 5 silicon atoms. Linear volatile silicone materials generally have viscosities less than about 5 centistokes at 25° C., whilst cyclic materials typically have viscosities of less than about 10 centistokes.

Non-volatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile silicone emollients useful in the present compositions are the polydimethyl siloxanes having viscosities from about 10 to about 400 centistokes at 25° C. Among the ester emollients are:
(1) Alkenyl or alkyl esters of fatty acids having 10 to 20 carbon atoms. Examples thereof include isoarachidyl neopentanoate, isononyl isononanoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, myristyl myristate, stearyl stearate and arachidyl behenate.
(5) Sterol esters of which cholesterol fatty acid esters are examples thereof. Humectants of the polyhydric alcohol type may also be employed as part of the cosmetic vehicle in cosmetic compositions, especially skin care compositions of this invention. The humectant aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. The amount of humectant may range anywhere from 0.5 to 30%, preferably between 1 and 15% by weight of the final skin care composition.

Compositions according to the invention may beneficially comprise little or no additional thickener. However, if desired, thickeners (in minor amounts) may also be utilised as part of the cosmetically acceptable vehicle of compositions according to the present invention. Suitable thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotiuin, carrageenan, pectin and combinations of these gums. Amounts of the thickener may range from 0.0001 to 2%, usually from 0.001 to 1%, by weight of the composition, if at all.

Compositions according to the invention may additionally comprise minor amounts of other surfactants.

An oil or oily material may be present, together with an emulsifier to provide typically an oil-in-water emulsion, though this will depend largely on the average hydrophilic-lipophilic balance (HLB) of the emulsifier employed.

Various types of additional active ingredients may be present in cosmetic compositions of the present invention. Actives are defined as (skin) benefit agents other than emollients and other than ingredients that merely improve the physical characteristics of the composition. Although not limited to this category, general examples include additional anti-sebum ingredients such as talcs and silicas, and sunscreens.

Sunscreens include those materials commonly employed to block ultraviolet light. Illustrative compounds are the derivatives of PAA, cinnamate and salicylate. For example, azobenzophenone (Parsol 1789®) octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks Parsol MCX and Benzophenone-3 respectively. The exact amount of sunscreen employed in the compositions can vary depending upon the degree of protection desired from the sun's UV radiation.

Many cosmetic compositions, especially those containing water, must be protected against the growth of potentially harmful micro-organisms. Preservatives may therefore be necessary, though because of the essentially anhydrous nature of the concentrate, the need for preservatives may be reduced.

Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the final skin care composition.

Compositions according to the invention may also include a retinoid. Retinoids increase collagen synthesis by dermal fibroblasts. This results in protection from sun damage and smoothening of wrinkled skin. The term “retinoids” as used herein, includes retinoic acid, retinol, retinal, and retinyl esters. Included in the term “retinoic acid” are 13-cis retinoic acid and all-trans retinoic acid.

The term “retinol” as used herein includes the following isomers of retinol: all-trans-retinol, 13-cis-retinol, 11-cis-retinol, 9-cis-retinol, 3,4-didehydro-retinol. Preferred isomers are all-trans-retinol, 13-cis-retinol, 3,4-didehydro-retinol, 9-cis-retinol. Most preferred is all-trans-retinol, due to its wide commercial activity.

Retinyl ester is an ester of retinol. The term “retinol” has been defined above. Retinyl esters suitable for use in the present invention are C₁-C₃₀ esters of retinol, preferably C₂-C₂₀ esters, and most preferably C₂, C₃, and C₁₆ esters because they are more commonly available. Examples of retinyl esters include but are not limited to: retinyl palhnitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl laurate, retinyl tridecanoate, retinyl myristate, retinyl pentadecanoate, retinyl heptadecanoate, retinyl stearate, retinyl isosterate, retinyl nonadecanoate, retinyl arachidonate, retinyl behenate, retinyl linoleate, retinyl oleate, retinyl lactate, retinyl glycolate, retinyl hydroxy caprylate, retinyl hydroxy laurate, retinyl tartrate.

If present, the retinoids in the present invention may be present in an amount of from 0.001% to 10%, preferably from 0.01% to 1%, and most preferably from 0.01% to 0.05% by weight of the final skin care composition.

The invention will now be further demonstrated by way of example only.

### EXAMPLES

Examples 1-4 demonstrate the preparation of suitable base materials for use in topical cosmetic compositions.

Weight (g)

| Ingredients | 1 | 2 | 3 | 4 |
|---|---|---|---|---|
| Fatty Acid* | 1.8 | 1.8 | 1.8 | 1.8 |
| NaOH | 0.033 | — | — | — |
| KOH | — | 0.0466 | — | — |
| Triethanolamine | — | — | 0.125 | — |
| Sodium Stearate | — | — | — | 0.254 |
| Water | 8.2 | 8.2 | 8.2 | 8.0 |

*Pristerene 4911, a commerical blend of approximately 50:50 stearic:palmitic acids.

### Example 5

| Ingredients | w/w % of the topical composition |
|---|---|
| Glycerine | 1 |
| Fatty acid* | 18.0 |
| Cetyl alcohol | 0.5 |
| Potassium hydroxide | 0.5 |
| Octyl methoxy cinnamate | 1.0 |
| Butyl methoxy dibenzoyl | 0.5 |
| Dimethicone | 0.2 |
| Niacinamide | 1 |
| Tetra sodium EDTA | Trace |
| Methyl paraben | 0.1 |
| Propyl paraben | 0.1 |
| Phenoxy ethanol | 0.4 |
| Perfume | 0.5 |
| Aqua | to 100 |

All of the compositions in example 1-5 were mixed in the form of a powder or tablet according to one or more of the methods outlined below. On the provision of heated water the product re-constituted to form in the case of examples 1-4 a satisfactory composition base, and in the case of in the example 5 a satisfactory topical product.

Methods of Making the Compositions

1. Hand Shaking

Powder or tablet made from any of formulations 1 to 5 (excluding water) was put in a screw top glass vial (ca 25 ml volume). Boiling water was then added inside the vial to bring the total weight in the vial up to the level required for the fall formulation, and the cap was replaced to seal the vial. The vial was then shaken for about 100 seconds, and then cooled down in an ambient environment or in a refrigerator. The total cooling time was about 3-5 minutes.

2. Vortex Mixer

Powder or tablet made from any of formulations 1 to 5 (excluding water) was put in a glass vial (ca 25 ml volume). Boiling water was then added inside the vial, and the cap was replaced to seal the vial. This vial was then mixed by a vortex mixer for about 100 seconds. The vibration speed of the vortex mixer ranged from 500-3000 rpm. The vial was then cooled in ambient environment or in a refrigerator. The total cooling time was about 3-5 minutes.

3. Ultrasonic Heating and Mixing

Powder or tablet made from formulation 1-5 was put in a plastic container. NaOH and water was then added to the container at ambient temperature, (though the example would function equally satisfactorily if the NaOH was pre-prepared with the powder or tablet, and simply hot water added, or if the sodium hydroxide was pre-combined with the fatty acid, and cold water subsequently added prior to heating). An ultrasonic heating and mixing device was used to heat the contents of the container to 80° C. in 60 seconds. It was then cooled in ambient environment or in a refrigerator. The total cooling time was about 3-5 minutes.

4. Microwave Heating and Hand Shaking

Powder or tablet made from any of formulations 1 to 5 (excluding water) was put in a glass vial (ca 25 ml volume). Tap water was then added to the vial, and the cap was replaced to seal the vial. The vial was placed in a microwave and heated 15-25 seconds at 800 W power. The vial was then shaken by hand for about 100 seconds, and then cooled in ambient environment or in a refrigerator. The total cooling time was about 3-5 minutes.

1. A method of providing a topical base composition for use in the preparation of a cosmetic composition, especially a topical skin care composition, comprising:
(a) providing a cosmetic container;
(b) providing in the container an effective amount of a fatty acid material having a melting point in the range of 40° C. to 80° C.; and other components necessary to form a fatty acid soap material which is substantially anhydrous, wherein the substantially anhydrous composition is a simple mechanical mixture;
(c) providing sufficient heated water to the container such that substantially all of the fatty acid material is solubilised to provide a fatty acid soap material; and
(d) agitating the contents of the container;
whereby a cream or lotion base is formed.

2. A method according to claim 1, wherein the method is carried out in a cosmetic container by a consumer who seals the cosmetic container just prior to agitation.

3. A method according to claim 1, wherein the fatty acid soap material is provided in the container by pre-dosing the container with a mixture of a soap and a fatty acid material.

4. A method according to claim 1, wherein the fatty acid soap material is provided in the container by pre-dosing the container with a mixture of fatty acid material and an effective amount of an organic or inorganic base.

5. A method according to claim 4, wherein the base is an inorganic base which is a hydroxide material.

6. A method according to claim 1, wherein the fatty acid material comprises a fatty acid blend.

7. A method according to claim 6, wherein the fatty acid blend comprises a mixture of fatty acid moieties having chain lengths of C₈ to C₂₂.

8. A method according to claim 7, wherein the fatty acid blend comprises any of pelargonic, lauric, myristic, palmitic, stearic, isostearic, oleic, linoleic, ricinoleic, arachidic, behenic or erucic acids.

9. A method according to claim 8, wherein the fatty acid blend comprises at least 50% of stearic and/or palmitic acids.

10. A method according to claim 1 wherein the resulting ratio of soap to fatty acid material after the provision of the heated water is in the ratio 1:1 to 1:20, more preferably 1:2 to 1:10.

11. A method according to claim 1 wherein the heated water has a temperature of at least 85° C., more preferably at least 95° C.

12. A method according to claim 1 wherein the container additionally contains other components necessary to provide a topical skin care cream or lotion.

13. A method according to claim 1 wherein ingredients of the fatty acid soap material are provided in the container in the form of a tablet, dried powder or amorphous mass.

14. A re-sealable cosmetic container containing an anhydrous composition comprising a simple mechanical mixture of a fatty acid material having a melting point in the range 40° C. to 80° C. and an organic or inorganic base.

15. A re-sealable cosmetic container containing an anhydrous composition comprising a simple mechanical mixture of a fatty acid material having a melting point in the range 40° C. to 80° C. and a soap.

16. A re-sealable cosmetic container according to claim 14 additionally containing other components necessary to provide a skin care cream or lotion.

17. An anhydrous composition comprising a simple mechanical mixture of a fatty acid material having a melting point in the range 40° C. to 80° C. and a soap in the form of a tablet, powder or amorphous mass for dosing into a container for use according to claim 1.
